(19) United States
(12) Patent Application Publication
(10) Pub. No.: US 2019/0018932 A1
(43) Pub. Date: Jan. 17, 2019

(54) CONSUMPTION ADVISOR COMPUTING SYSTEM AND METHOD

(71) Applicant: MasterCard International Incorporated, Purchase, NY (US)

(72) Inventors: Peter Groarke, Dublin (IE); Ahmed Hosny, Dublin (IE); Ross Phelan, Kildare (IE); Rohit Gupta, Dublin (IE)

(21) Appl. No.: 15/650,636

(22) Filed: Jul. 14, 2017

(51) Int. Cl. G06F 19/00; A61B 5/00; G06Q 30/06; G06Q 20/32

(52) U.S. Cl. CPC G06F 19/3475; A61B 5/0002; G06Q 20/32; G06Q 30/0633; G06Q 30/06

(57) ABSTRACT

A consumption digestion advice (CDA) tracker computing device includes a processor coupled in communication with a memory. The processor is programmed to receive user transaction data from a payment network for a selected user. In addition, the processor is programmed to retrieve food, medicine, and/or condition contraindication data from a third party database for the selected user. The processor is also programmed to process the transaction data and the food, medicine, and/or condition contraindication data to determine a personalized indicator indicating at least one contraindication relating to the transaction data. Moreover, the processor is programmed to transmit the personalized indicator to a user computing device for display.

### BACKGROUND

This disclosure relates to consumer health tracking and, more specifically, to a consumption advisor computing system and method that is configured to track food, beverage, and/or medicine intake by analyzing food, beverage and/or medicine purchases, and provide advice about food-related and medicinal purchases.

Many consumers suffer from occasional and/or chronic digestive conditions, such as, acid reflux, heartburn, Gastroesophageal Reflux Disease (GERD), Irritable Bowel Syndrome (IBS), and the like. At least some of these consumers will visit a doctor to try to identify the condition, particularly when the symptoms interfere with their normal daily routines. In addition, some of these consumers may find relief from such conditions by avoiding trigger foods and/or beverages that trigger their symptoms, and/or by taking over-the-counter antacids or other medications that may help reduce stomach acid production and/or inflammation of the digestive tract. However, identifying the trigger foods and/or beverages may not always be that easy.

Often, doctors when confronted with such digestive problems may ask their patients to maintain a journal of the food and/or beverages that the consumer consumes over a period of time, such as, a month or longer. The consumer is also asked to record any negative symptoms experience during this period. The doctor can review the journal and prescribe palliative options, such as antacids and/or other prescription medications. In addition, the doctor may identify certain trigger foods and prescribe a particular diet and/or suggest certain foods and/or beverages to avoid. Journaling, however, can be difficult and may not help sufficiently identify such digestive problems and/or trigger foods and/or beverages.

Consumers may use various food journaling services or programs, in which the user manually logs their food and beverage consumption. The user must enter each and every food consumed in a particular meal or particular day, including the amount of each food consumed, which can be a difficult task to complete accurately. As a result, many users over or underestimate their food consumption, do not enter all the food consumed, and many users get tired of the tedious task of entering food into their journal, which leads to the user abandoning the use of the service entirely. This issue may be particularly relevant to users whose symptoms are occasional, such as when the symptoms do not interfere with their normal daily routine.

### BRIEF DESCRIPTION OF THE DISCLOSURE

In one aspect, a consumption digestion advice (CDA) tracker computing device is provided. The CDA tracker computing device includes a processor coupled in communication with a memory. The processor is programmed to receive user transaction data from a payment network for a selected user. In addition, the processor is programmed to retrieve food, medicine, and/or condition contraindication data from a third party database for the selected user. The processor is also programmed to process the transaction data and the food, medicine, and/or condition contraindication data to determine a personalized indicator indicating at least one contraindication relating to the transaction data. Moreover, the processor is programmed to transmit the personalized indicator to a user computing device for display, the user computing device associated with the selected user.

In another aspect, a consumption digestion advice (CDA) tracker computing device is provided. The CDA tracker computing device includes a processor coupled in communication with a memory. The processor is programmed to receive user transaction data from a payment network for a selected user, and retrieve food, medicine, and/or condition contraindication data from a third party database for the selected user. In addition, the processor is programmed to process the transaction data to determine at least a first purchased product and a second purchased product. Moreover, the processor is programmed to determine that the first and second purchased products are related to the at least one of a health condition and a symptom based on the food, medicine, and/or condition contraindication data.

In yet another aspect, a computer-implemented method for tracking food-related transactions of a user and providing advice about food-related and medicinal purchases is provided. The method uses a CDA tracker computing device that includes a processor coupled in communication with a memory. The method includes receiving user transaction data from a payment network for a selected user, and retrieving food, medicine, and/or condition contraindication data from a third party database for the selected user. In addition, the method includes processing the transaction data and the food, medicine, and/or condition contraindication data to determine a personalized indicator indicating at least one contraindication relating to the transaction data. Moreover, the method includes transmitting the personalized indicator to a user computing device for display, the user computing device associated with the selected user.

In still another aspect, at least one non-transitory computer-readable storage media having computer-executable instructions embodied thereon is provided. When executed by at least one processor, the computer-executable instructions cause the processor to receive user transaction data from a payment network for a selected user, and retrieve food, medicine, and/or condition contraindication data from a third party database for the selected user. In addition, the computer-executable instructions cause the processor to process the transaction data and the food, medicine, and/or condition contraindication data to determine a personalized indicator indicating at least one contraindication relating to the transaction data. Moreover, the computer-executable instructions cause the processor to transmit the personalized indicator to a user computing device for display, the user computing device associated with the selected user.

### BRIEF DESCRIPTION OF THE DRAWINGS

FIGS. 1-9 show example embodiments of the methods and systems described herein.

FIG. 1 is a block diagram of a consumption digestion advice (CDA) tracking system including a CDA tracker computing device (“CDA tracker”).

FIG. 2 is a schematic diagram illustrating an example multi-party payment card system in communication with the CDA system 100 shown in FIG. 1.

FIG. 3 illustrates an example configuration of a client computing device shown in FIG. 1.

FIG. 4 illustrates an example configuration of a server system shown in FIG. 1.

FIG. 5 is a component view of an example CDA tracker shown in FIG. 1 that may be used in the CDA system shown in FIG. 1.

FIG. 6 is a schematic diagram of the process of a recommendation component of the CDA tracker shown in FIG. 1.

FIG. 7 is a schematic diagram of the process of a correlating component of the CDA tracker shown in FIG. 1.

FIG. 8 is a flowchart of a method for tracking food-related transactions of a user and providing advice about food-related and medicinal purchases using the CDA system shown in FIG. 1.

FIG. 9 is a flowchart of another method for tracking food-related transactions of a user and providing advice about food-related and medicinal purchases using the CDA system shown in FIG. 1.

Like numbers in the Figures indicates the same or functionally similar components. Although specific features of various embodiments may be shown in some figures and not in others, this is for convenience only. Any feature of any figure may be referenced and/or claimed in combination with any feature of any other figure.

### DETAILED DESCRIPTION OF THE DISCLOSURE

The consumption digestion advice (CDA) system described herein is configured to advise a user about their food-related and medicinal purchases. In particular, the CDA system is configured to track a user's food-related and medicinal purchases and/or collect a user's medical conditions and/or symptoms to make food-related purchase recommendations to the user. The CDA system includes a consumption digestion advice computing device (“CDA tracker”) in communication with a transaction processor that is configured to process payment transactions, and/or a database that is configured to store data related to the transactions (i.e., “transaction data”). The transactions are associated with purchases made by cardholders using payment cards, and are processed over a payment network that includes the transaction processor and/or the database. The CDA tracker includes a processor coupled in communication with a memory. The CDA tracker is further coupled in communication with a database for storing information, such as, for example, a user's health data (e.g., medical conditions and/or symptoms), transaction data associated with food-related and/or medicinal purchase transactions initiated by the user and/or by other cardholders, and food, medicine, and/or condition contraindication data.

The CDA tracker is further in communication with one or more user computing devices (e.g., a smart phone, laptop, desktop, tablet, etc.), and is configured to receive line item data used to identify a user's food-related and medicinal purchases. The line item data corresponds to the user's transaction data. More specifically, each line item of the line item data corresponds to a specific product purchased by the user, as indicated by the user's transaction data.

In one example embodiment, the CDA tracker is also configured to receive user health data related to a user's medical conditions and/or symptoms from one of the user computing devices. As used herein, “user health data” refers generally to data that may impact a user's daily and overall health, such as, but not limited to, identified medical conditions, known allergies, dietary restrictions (e.g., Kosher, Halal, vegan, low salt), and/or symptoms experienced by the user.

One or more user computing device(s) associated with the user may track the user's health data, or may receive input associated therewith. At least some of the data in a user profile (e.g., health data) may be input directly or manually by the user, and at least some of the user profile may be automatically tracked, stored, updated, edited, or accessed by the user computing device(s). For example, for one user: (i) the user may track their fitness using a fitness wearable, which automatically updates certain health data for the user, such as an activity level, caloric output, and/or heart rate; (ii) the user may track their weight using a smart scale, which automatically updates health data for the user, such as a weight, Body Mass Index (BMI), and/or body composition; and (iii) the user may track their food consumption using an application on their smart phone, which may enable the user to scan barcodes of consumed items and add those items to their health data (e.g., caloric intake, food log, etc.). The CDA tracker is configured to enable access from a plurality of user computing device(s) to a user's profile, to make tracking and monitoring the user's health data more efficient and less burdensome for the user.

In the example embodiment, the user may be required to manually set up their user profile using the user computing device, wherein “manually set up” includes filling in a number of fields with information associated with the user. In one embodiment, at least some of the user profile may be imported from another source, such as from a health record from the user, for example, from the user's healthcare or health insurance provider and/or from a user profile associated with another application (e.g., a fitness application or a social media application).

The CDA tracker may receive line item data from a number of sources, including for example, a credit or debit card financial transaction message on a payment network. The financial transaction message can include product-specific-data, such as stock keeping unit (SKU) data. In addition, the CDA tracker may receive line item data from payment gateways through which the payment may be routed. The payment gateways may have an alternative product/goods identifier other than SKU data. Moreover, the CDA tracker can receive line item data from one of the user computing devices, such as by QR/bar code scanning, when the cardholder is shopping on a Masterpass enabled merchant site, or from a list of items in a virtual shopping cart. In some embodiments, the CDA tracker may be able to identify the purchased food and/or medications by statistical analysis of the purchase amounts.

The CDA tracker is also in communication with one or more external or third party services/open databases for receiving food, medicine, and/or condition contraindication data. Such external services/open databases provide data related to medical conditions, symptoms, medicines, and foods, and the associated indications and contraindications of such items. In the example embodiment, the CDA tracker leverages the user's health data, their purchase history, and food, medicine, and/or condition contraindication data to advise a user about their food-related and medicinal purchases and/or make purchase recommendations to the user.

In the example embodiment, a user opts in to use the CDA tracker service with, for example, a MASTERPASS account. (Masterpass is a registered trademark of Mastercard International Incorporated.) The user may choose to input the user's health data, such as any known medical conditions, allergies, and/or symptoms, into a user profile. The CDA tracker will present options to the user to describe their allergies and/or medical conditions. In some embodiments, the CDA tracker may be able to pre-populate portions of the user health data using the line item data for the user's historical purchase data, if available. In addition, in some embodiments, the CDA tracker may provide an option for the user to input their doctor contact information and/or their medical insurance provider information. It is noted that inputting user health data is not required for the CDA tracker to advise a user about their food-related and medicinal purchases. Rather, the user's health data is used to provide more targeted advice.

The CDA tracker is configured to analyze the user's historical transactions to identify the foods and medicines purchased by the user. The purchases of specific products can be associated with a purchase frequency. In some embodiments, the CDA tracker may be able to determine probable medical conditions based on the user's historical food and medicine purchases. For example, the CDA tracker may identify occasional purchases of antacid. Based on food, medicine, and/or condition contraindication data received from the external or third party services/open databases described above, the CDA tracker associates antacid with acid reflux. The CDA tracker may cross-reference the antacid with known contra-indicated foods. As such, the CDA tracker may identify a purchase of contra-indicated foods and correlate the purchase of antacid with a purchase of avocados, for example, which are a known risk factor for acid reflux. Thus the CDA tracker may provide an alert to the user to advise a user about the correlation of avocados to acid reflux.

In some embodiments, the user can input specific bouts of symptoms to the CDA tracker via a notification API, for example. The CDA tracker may then analyze the user's recent purchases to determine a food, beverage, and/or medicine that may be a contra-indicator of such symptoms. In addition, the user can input specific occasions when a medicine successfully treated a particular symptom. The CDA tracker maintains this information to facilitate improving advice a user about their food-related and medicinal purchases.

In the example embodiment, after the user opts in to use the CDA tracker service, the CDA tracker provides a seamless user experience with, for example, Masterpass enabled applications, services, and/or merchants. For example, when the user is shopping on a MASTERPASS enabled merchant site, the user will receive personalized indicators on the items the user is browsing. The personalized indicators will specify whether an item is safe or if it has any adverse side effects to the user's medical conditions/symptoms. For example, the CDA tracker may correlate a user's purchase of antacid with the purchase of curry, and determine that the user suffers from acid reflux. When the user adds curry to his shopping cart (whether virtual or by scanning the item with the user's computing device), the CDA tracker may provide a personalized indicator noting, for example, that 75% of sufferers of same condition using the CDA tracker service experienced an improvement when they stopped eating curry or took an antacid. In some embodiments, the user may select the indicator to be provided with additional details that may be personalized to the user.

The CDA tracker is also configured to provide potential drug interaction notifications and/or contraindication information related to a purchased medicine and a user's medical condition or symptoms. For example, a user's health information indicates that the user is suffering from a stomach ulcer. When the user purchases aspirin, for example, for a headache, the CDA tracker may provide an indicator identifying that aspirin is contraindicated in users with ulcers. In some embodiments, where other users have input specific occasions when a medicine successfully treated a particular symptom, the CDA tracker may use this information to provide a recommendation to the user. For example, the CDA tracker may provide an indicator to the user recommending acetaminophen rather than aspirin based on feedback from other users and the food, medicine, and/or condition contraindication data.

In one embodiment, the user may request personalized medical advice and/or prescriptions on the user's medical condition or symptoms they have linked to a food they consumed earlier. For example, the CDA tracker may receive the request from the user and provide additional information on the user's condition based on the food, medicine, and/or condition contraindication data available from the external or third party services/open databases.

In an alternative embodiment, the user may be able to send their food diaries seamlessly to their doctor or healthcare provider. Healthcare providers may choose to opt-in to the CDA tracker service to receive immediate access to their patient's food diaries, and the food and medicines they consume. This may facilitate the healthcare provider attaining a better understanding of their patient's health. In other embodiments, the user will also be able to share their fitness level with their medical insurance providers to secure any available discounts for providing such information.

The CDA tracker is further in communication with at least one merchant computing device. The merchant computing device may include a point-of-sale (POS) device or terminal at a merchant location (e.g., a brick-and-mortar merchant location) and/or may include a virtual merchant POS (e.g., for a merchant with online purchase functionality). In the example embodiment, each merchant computing device is associated with a merchant, such as a grocery store or other vendor having food-related goods and/or services available for purchase.

The CDA tracker is configured to communicate with the one or more merchant computing device(s) to receive line item data concerning the goods to be purchased. For example, integrated merchants will send line item data to the CDA tracker when the user selects the option to pay. The CDA tracker analyzes the items and provides any individualized indicators to the user. For example, the CDA tracker will be able to provide the individual indicators to the user through the merchant computing device. In the example embodiment, the merchants do not receive the user's health data. Rather, through integration with MASTERPASS, for example, the merchant will pass the item's nutritional information to the CDA tracker and the CDA tracker will be responsible for managing the indicators or warnings.

In the example embodiment, the CDA tracker is configured to access or communicate with the one or more merchant computing devices through a cloud-based Application Programming Interface (API), which functions as a middleware layer and facilitates transmission of transaction data from a merchant computing device to the payment network. The API further enables any number of merchant computing device to communicate with the CDA tracker and, accordingly, with the user through the user computing device and an e-commerce application. Additionally, the API enables the user to access and interact with certain features of the merchant computing device(s) through the e-commerce application, including making mobile payments directly to a merchant POS device. In one embodiment, the user may only be permitted to use the e-commerce application to make payments to a merchant POS device when the user is in a specific proximity to the merchant POS device (e.g., the user is in a grocery store and may purchase their groceries through the e-commerce application). In other embodiments, the user may be permitted to initiate transaction with the merchant POS device from any location (e.g., the user is making a payment for delivery from a merchant through the e-commerce application at a location remote from the merchant's location).

In one embodiment, the CDA tracker is further configured to advise a user about their food-related purchases and/or make purchase recommendations to the user in a restaurant setting as well, for example, when a user is dining out. The CDA tracker is configured to advise the user concerning ingredients and/or recipes based on the user's health data, historical purchases, and/or food, medicine, and/or condition contraindication data.

In one embodiment, the CDA tracker is in communication with at least one of (i) a transaction processor that is configured to process payment transactions, and/or (ii) a database that stores data related to the transactions (“transaction data”). As described above, in the example embodiment, the transaction processor and the database are part of a payment processing network that is configured to process payment transactions, such as for credit and debit cards. Transaction data includes such elements as a transaction amount, a merchant identifier, and a description of the purchase made (e.g., a particular food item or product). In some embodiments, transaction data may further include additional elements such as a location identifier, which may identify where the transaction was initiated (i.e., a location of the consumer), and/or the location of the merchant. The CDA tracker receives transaction data associated with purchases of food-related and/or medicinal items, made by the user using their payment card. The CDA tracker may associate and/or index the transaction data with the user profile for the user, such that the transaction data for the user is only stored and/or processed in conjunction with that user's profile. The CDA tracker stores and/or processes the transaction data to determine which items were purchased, from which merchants the items were purchased, and/or which brands of items were purchased. Accordingly, the CDA tracker develops a usage history associated with the user profile such that the CDA tracker may generate intelligent advice and/or recommendations for the user that accord with their actual item usage preference (e.g., brands, merchants, items).

In some embodiments, the CDA tracker is further in communication with a computing device associated with a service provider, which provides a health-related service to the user. For example, the service provider may be a healthcare provider or an insurance provider. The CDA tracker may send health reports to the service provider that includes the user's health data and other data from the user's profile. The CDA tracker updates the service provider on how well or how poorly the user is meeting their health goals, or whether the user is maintaining healthy habits and behavior. The service provider may provide rewards to the user based on the health reports. For example, the insurance provider may provide the user a discount on their health insurance premium or a “cash back” incentive.

In the example implementation, any information stored on the CDA tracker system does not include any personally identifiable information (PII), but rather includes analyzed, anonymized, and/or aggregated data that does not specifically identify a consumer (e.g., a cardholder) that initiated a transaction. In other embodiments, where the CDA tracker system may store PII, any stored PII is encrypted and/or otherwise secured. Moreover, in any embodiments in which PII may be collected, the consumer from which the PII may be collected is provided an opportunity to agree to or deny collection of such data.

The technical effects of the systems and methods described herein are achieved by performing at least one of the following steps: (a) receiving user transaction data from a payment network; (b) retrieving food, medicine, and/or condition contraindication data from a third party open database; (c) processing the transaction data and the food, medicine, and/or condition contraindication data to determine a personalized indicator indicating at least one contraindication relating to the transaction data; (d) processing the transaction data to determine at least a first purchased product and a second purchased product; (e) determining that the first purchased product and the second purchased product are related to at least one of a common health condition and a common symptom based on the food, medicine, and/or condition contraindication data, and (f) transmitting the personalized indicator to a user computing device for display.

As used herein, the terms “transaction card,” “financial transaction card,” and “payment card” may include any suitable transaction card, such as a credit card, a debit card, a prepaid card, a charge card, a membership card, a promotional card, a frequent flyer card, an identification card, a prepaid card, a gift card, and/or any other device that may hold payment account information, such as mobile phones, smart phones, personal digital assistants (PDAs), key fobs, and/or computers. Each type of transactions card can be used as a method of payment for performing a transaction.

The term “processor,” as used herein, includes any programmable system including systems using central processing units, microprocessors, microcontrollers, reduced instruction set circuits (RISC), application specific integrated circuits (ASIC), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution mobile devices, clusters, personal computers, workstations, clients, servers, and a processor wherein the memory includes RAM memory, ROM memory, EPROM memory, EEPROM memory, and non-volatile RAM (NVRAM) memory. The above memory types are exemplary only, and are thus not limiting as to the types of memory usable for storage of a computer program.

Furthermore, as used herein, the term “real-time” includes at least one of the times of occurrence of the associated events, the time of collection of data, the time to process the data, and the time of a system response to the events and the environment. In the embodiments described herein as occurring in real-time, these activities and events occur substantially instantaneously.

In one embodiment, a computer program is provided, and the program is embodied on a computer-readable medium. In an example embodiment, the CDA system is executed on a single computer system, without requiring a connection to a sever computer. In a further example embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of AT&T located in New York, N.Y.). The application is flexible and designed to run in various different environments without compromising any major functionality. In some embodiments, the CDA system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

The following detailed description illustrates embodiments of the disclosure by way of example and not by way of limitation. It is contemplated that the disclosure has general application to processing purchase patterns in industrial, commercial, and residential applications.

As used herein, an element or step recited in the singular and preceded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

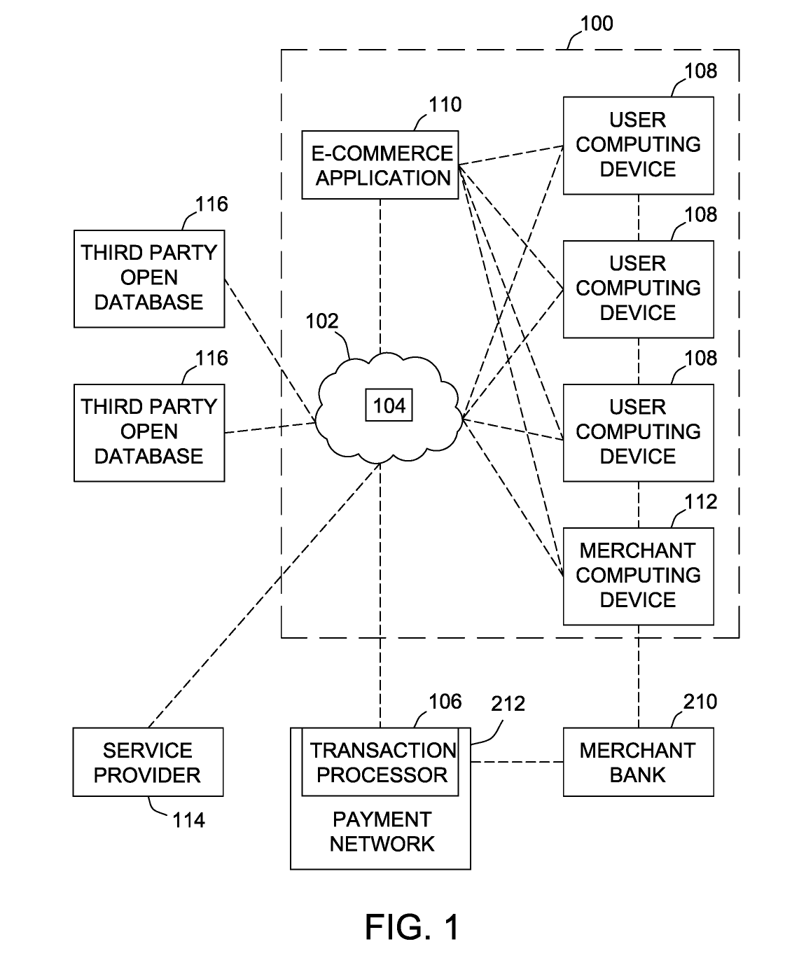

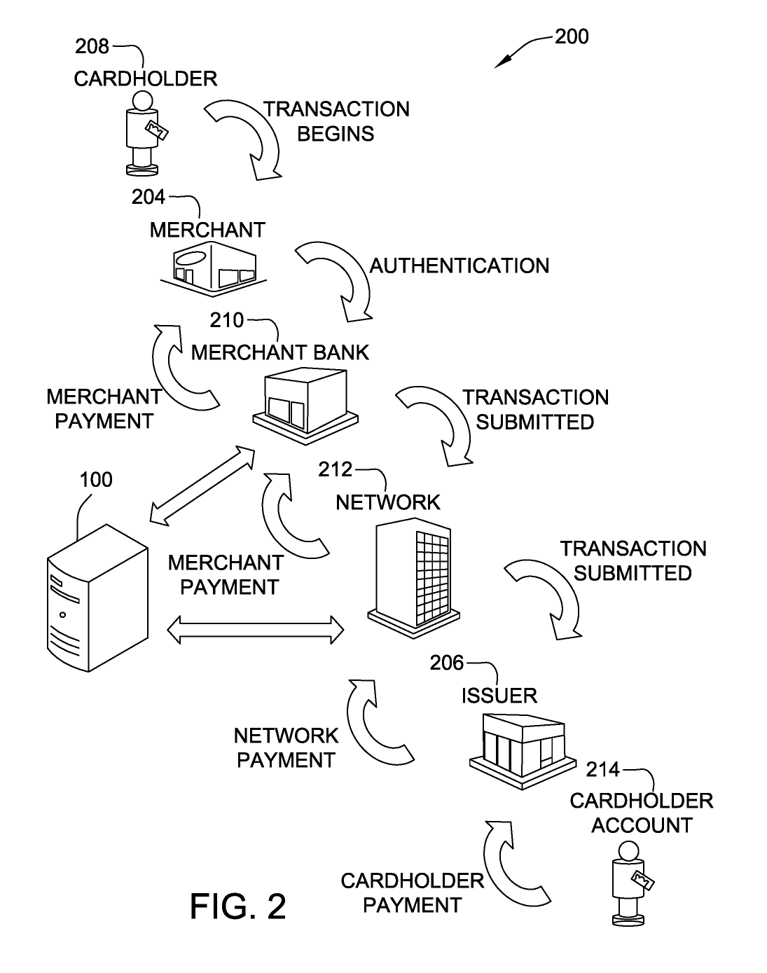

FIG. 1 is a block diagram of a consumption digestion advice (CDA) tracking system 100 including a CDA tracker computing device (“CDA tracker”) 102. CDA tracker 102 includes at least one processor (not shown) in communication with a database 104. Database 104 contains information on a variety of matters, including stored transaction data for one or more users, line item data used to identify one or more users food-related and medicinal purchases, one or more user profiles, and other information described herein. In one embodiment, database 104 is stored on CDA tracker 102. In an alternative embodiment, database 104 is stored remotely from CDA tracker 102 and may be non-centralized. In the example embodiment, CDA system 100 is in communication with a transaction processor 106, which is integral to and/or associated with a payment network 212. Payment network 212 is described more fully herein with respect to FIG. 2.

In the example embodiment, CDA system 100 further includes a plurality of client subsystems, also referred to as client systems or user computing devices 108. In one embodiment, user computing devices 108 are computers including a web browser, such that CDA tracker 102 is accessible to user computing devices 108 using the Internet. User computing devices 108 are interconnected to the Internet through many interfaces including a network, such as a local area network (LAN) and/or a wide area network (WAN), dial-in connections, cable modems, wireless-connections, and special high-speed ISDN lines. User computing devices 108 may be any device capable of interconnecting to the Internet including a mobile computing device, such as a laptop or desktop computer, a web-based phone (e.g., a “smart phone”), a personal digital assistant (PDA), a tablet or phablet, a fitness wearable device, a web-connectable appliance, a “smart watch” or other wearable device, or other web-connectable equipment. Although three user computing devices 108 are shown in FIG. 1, it should be understood that CDA system 100 may include any number of user computing devices 108.

CDA tracker 102 is configured to communicate with a user computing device 108 associated with a user (not shown in FIG. 1). User computing device 108 is configured to execute for display on an electronic commerce (e-commerce) application 110, for example, Groceries™ by Mastercard. In some embodiments, the e-commerce application 110 may be stored in a cloud-based interface, which may include cloud storage capability as well as any cloud-based API that facilitates communication between a merchant computing device 112 and the CDA tracker 102 and/or between user computing devices 108 and the CDA tracker 102. The e-commerce application 110 stores a user profile associated with the user, for example, in database 104. The user profile includes user health data for the user. Additionally, the user profile may be viewed, accessed, and/or updated by user computing devices 108. The user accesses the e-commerce application 110 to communicate with CDA tracker 102, in particular, to request and receive advice and/or purchase recommendations from CDA tracker 102.

CDA system 100 further includes merchant computing device 112, which may include a real or virtual point-of-sale (POS) device, an inventory computing device, or any other computing device capable of communicating with transaction processor 106 and/or with CDA tracker 102. In the example embodiment, merchant computing device 112 is associated with a merchant (not shown). CDA tracker 102 is configured to access merchant computing device 112 through, for example, a cloud-based interface. CDA tracker 102 is configured to communicate with merchant computing device 112 to access data (e.g., line item data, SKU information, etc.) and/or to access any virtual merchant capabilities of the merchant (e.g., to order delivery of an item from the merchant). Additionally or alternatively, at least one of user computing devices 108 may access merchant computing device 112 directly, using the e-commerce application 110 as an interface, to access the virtual merchant capabilities of the merchant. Although only one merchant computing device 112 is shown in FIG. 1 for clarity, it is understood that CDA tracker 102 may be coupled in communication with any number of merchant computing devices 112.

In the example embodiment, CDA tracker 102 receives the user's historical transaction data from, for example, payment network 212. To identify the foods and medicines purchased by the user, the CDA tracker 102 may also receive line item data from the merchant computing device 112 and/or payment network 212. In the example embodiment, the CDA tracker 102 determines probable medical conditions based on the user's historical food and medicine purchases. The CDA tracker 102 may access one or more third party open databases 116 to retrieve food, medicine, and/or condition contraindication data. In addition, the CDA tracker may access database 104 to retrieve the user profile for the user. The user profile may include the user's health data. In some embodiments, the CDA tracker 102 receives real-time purchase data related to products the user is purchasing. For example, integrated merchants will send line item data to the CDA tracker 102 when the user selects the option to pay. The CDA tracker 102 analyzes the items and provides any individualized indicators to the user concerning the items to be purchased. In one embodiment, the CDA tracker 102 will be able to provide the individual indicators to the user through the merchant computing device 112 and/or the user computing device 108. The CDA tracker 102 leverages the user's health data, their purchase history data, and the food, medicine, and/or condition contraindication data to advise the user about their food-related and medicinal purchases and/or make purchase recommendations to the user. The CDA tracker 102 generates personalized indicators and returns the indicators to the user computing device 108 for display to the user within the e-commerce application 110.

In one embodiment, at least one user computing device 108 may be a fitness wearable (e.g., Fitbit® brand products, Jawbone® brand products, Garmin® brand products, or any other fitness wearable; Fitbit is a registered trademark of Fitbit, Inc., San Francisco, Calif.; Jawbone is a registered trademark of AliphCom, San Francisco, Calif.; Garmin is registered trademark of Garmin Ltd., Camana Bay, Cayman Islands). Fitness wearable 108 may refer generally to any wearable or semi-wearable computing device configured to track at least one health or fitness variable for the user (e.g., heart rate, activity, steps, routes, etc.). “Wearable” refers to devices and/or accessories that are attached to, coupled to, or otherwise worn on a user's person. “Semi-wearable” refers to devices and/or accessories that may utilize supplemental accessories or implements to be attached to, coupled to, or otherwise worn on a user's person, for example, a smart phone being held by the user or worn using a supplemental pack or band. Accordingly, fitness wearable 108 includes smart phones and/or other handheld devices that may have one or more fitness, health, or activity-tracking application installed thereon.

The user of fitness wearable 108 may pair fitness wearable 108 (or sync, or otherwise communicatively couple) with their user profile using the e-commerce application 110. Accordingly, the e-commerce application 110 may incorporate the user's activity (or other tracking fitness variable) into the user's health data. For example, if fitness wearable 108 tracks the user's activity for the day and reports that the user has burned 600 calories, a “credit” of 600 calories may be added to the user's health data for the day.

CDA tracker 102 is further in communication with at least one service provider 114. Service provider 114 represents any entity configured to provide a health-related service to the user. For example, and without limitation, service provider 114 may be a healthcare provider and/or a health insurance provider. CDA tracker 102 may send health reports to service provider 114 that include the user's health data and other data from the user's profile. Service provider 114 may provide rewards to the user based on the health reports. For example, a health insurance provider may provide the user a discount on their health insurance premium or a “cash back” incentive.

FIG. 2 is a schematic diagram illustrating an example multi-party payment card system 200 in communication with CDA system 100 shown in FIG. 1. CDA system 100 may be in communication with one or more elements of payment card system 200, such as, for example, a merchant bank 210 and a payment network 212. Payment card system 200 enables payment-by-card transactions for food-related and health-related purchases. The present disclosure relates to payment card system 200, such as a credit card payment system using the Mastercard® payment card system payment network 212 (also referred to as an “interchange” or “interchange network”). Mastercard® payment card system payment network 212 is a proprietary communications standard promulgated by Mastercard International Incorporated® for the exchange of financial transaction data between financial institutions that are customers of Mastercard International Incorporated®. (Mastercard is a registered trademark of Mastercard International Incorporated located in Purchase, N.Y.).

In payment card system 200, a financial institution such as an issuer 206 issues a payment account card, such as a credit card account or a debit card account, to a cardholder 208. Cardholder 208 uses the payment account card to tender payment for a purchase from a merchant 204. To accept payment with the payment account card, merchant 204 must normally establish an account with a financial institution that is part of the payment card system 200. This financial institution is usually called the “merchant bank,” the “acquiring bank,” the “acquirer bank,” or simply “acquirer.”

When cardholder 208 tenders payment for a purchase with a payment account card, merchant 204 requests authorization from merchant bank 210 for the amount of the purchase. The request may be performed over the telephone, but is usually performed through the use of a point-of-sale (POS) terminal, which may read the cardholder's account information from the magnetic stripe on the payment account card or EMV chip, or may accept the cardholder's account information electronically, and communicates electronically with the transaction processing computers of merchant bank 210. Alternatively, merchant bank 210 may authorize a third party to perform transaction processing on its behalf. In this case, the POS terminal will be configured to communicate with the third party. Such a third party is usually called a “merchant processor” or an “acquiring processor.” In some instances, a merchant (e.g., merchant 204) stores payment card information associated with a cardholder (e.g., cardholder 208) and requests authorization from merchant bank 210 using the stored payment card information, rather than reading the cardholder's account information from the payment card itself (i.e., a card-on-file (COF) transaction).

Using payment network 212 (e.g., using a transaction processor such as transaction processor 106, shown in FIG. 1), the computers of merchant bank 210 or the merchant processor will communicate with the computers of issuer 206, to determine whether the cardholder's account 214 is in good standing and whether the purchase is covered by the cardholder's available credit line or account balance. Based on these determinations, the request for authorization will be declined or accepted. If the request is accepted, an authorization code is issued to merchant 204.

When a request for authorization is accepted, the available credit line or available balance of cardholder's account 214 is decreased. Normally, a charge is not posted immediately to a cardholder's account 214 because bankcard associations, such as Mastercard International Incorporated®, have promulgated rules that do not allow a merchant to charge, or “capture,” a transaction until goods are shipped or services are delivered. When a merchant ships or delivers the goods or services, merchant 204 captures the transaction by, for example, appropriate data entry procedures on the POS terminal. If a cardholder cancels a transaction before it is captured, a “void” is generated. If a cardholder returns goods after the transaction has been captured, a “credit” is generated.

For PIN debit card transactions, when a request for authorization is approved by the issuer, the cardholder's account 214 is decreased. Normally, a charge is posted immediately to cardholder's account 214. The bankcard association then transmits the approval to the acquiring processor for distribution of goods/services, or information or cash in the case of an ATM.

After a transaction is captured, the transaction is cleared and settled between merchant 204, merchant bank 210, and issuer 206. Clearing refers to the communication of financial data for reconciliation purposes between the parties. Settlement refers to the transfer of funds between the merchant's account, merchant bank 210, and issuer 206 related to the transaction.

Transaction data associated with the transaction is processed by transaction processor 106 and/or is stored in a transaction database (not shown). More specifically, for transactions associated with food-related and/or health-related purchases, as described herein, transaction data may include such elements as a transaction amount, a merchant identifier, SKU data or other line item data, such as a description of the purchase made (e.g., a particular food item or product). In some embodiments, transaction data may further include additional elements such as a location identifier, which may identify where the transaction was initiated (i.e., a location of the consumer), and/or the location of the merchant. Transaction data is communicated between transaction processor 106 and CDA tracker 102 (shown in FIG. 1).

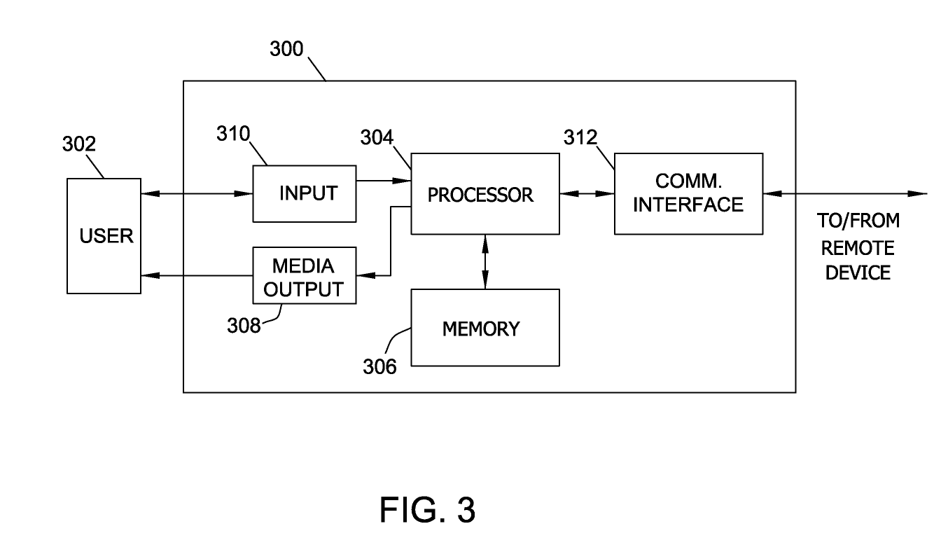

FIG. 3 illustrates an example configuration of a client computing device 300. Client computing device 300 may include, but is not limited to, user computing devices (“client systems”) 108 and/or merchant computing devices 112 (both shown in FIG. 1). Client computing device 300 includes a processor 304 for executing instructions. In some embodiments, executable instructions are stored in a memory 306. Processor 304 may include one or more processing units (e.g., in a multi-core configuration). Memory 306 is any device allowing information such as executable instructions and/or other data to be stored and retrieved. Memory 306 may include one or more non-transitory computer-readable media.

Client computing device 300 also includes at least one media output component 308 for presenting information to a user 302 (e.g., cardholder 208). Media output component 308 is any component capable of conveying information to user 302. In some embodiments, media output component 308 includes an output adapter such as a video adapter and/or an audio adapter. An output adapter is operatively coupled to processor 304 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, client computing device 300 includes an input device 310 for receiving input from user 302. Input device 310 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 308 and input device 310.

Client computing device 300 may also include a communication interface 312, which is communicatively coupleable to a remote device such as CDA tracker 102 or a web server operated by a merchant (e.g., merchant computing device 112, both shown in FIG. 1). Communication interface 312 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory 306 are, for example, computer-readable instructions for providing a user interface to user 302 via media output component 308 and, optionally, receiving and processing input from input device 310. A user interface may include, among other possibilities, a web browser and client application. Web browsers enable users 300 to display and interact with media and other information typically embedded on a web page or a website from a web server associated with a merchant. A client application allows users 300 to interact with a server application associated with, for example, a merchant and/or CDA system 100 (shown in FIG. 1).

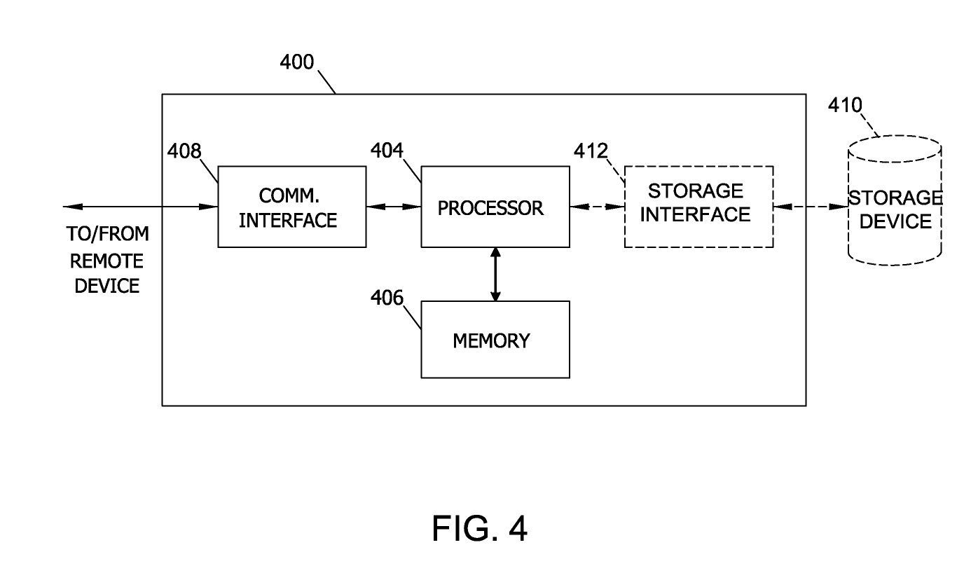

FIG. 4 illustrates an example configuration of a server system 400. Server system 400 may include, but is not limited to, CDA tracker 102, transaction processor 106, merchant computing device 112, service provider 114, and/or third party open databases 116 (all shown in FIG. 1). Server system 400 includes a processor 404 for executing instructions. Instructions may be stored in a memory 406, for example. Processor 404 may include one or more processing units (e.g., in a multi-core configuration).

Processor 404 is operatively coupled to a communication interface 408 such that server system 400 is capable of communicating with a remote device such as client computing device 300 or another server system 400. For example, communication interface 408 may receive requests from user computing devices 108 via the Internet, as illustrated in FIG. 1.

Processor 404 may also be operatively coupled to a storage device 410. Storage device 410 is any computer-operated hardware suitable for storing and/or retrieving data. In some embodiments, storage device 410 is integrated in server system 400. For example, server system 400 may include one or more hard disk drives as storage device 410. In other embodiments, storage device 410 is external to server system 400 and may be accessed by a plurality of server computing devices 402. For example, storage device 410 may include multiple storage units such as hard disks or solid state disks in a redundant array of inexpensive disks (RAID) configuration. Storage device 410 may include a storage area network (SAN) and/or a network attached storage (NAS) system.

In some embodiments, processor 404 is operatively coupled to storage device 410 via a storage interface 412. Storage interface 412 is any component capable of providing processor 404 with access to storage device 410. Storage interface 412 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 404 with access to storage device 410.

Memory 406 and 306 (shown in FIG. 3), and storage device 410 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

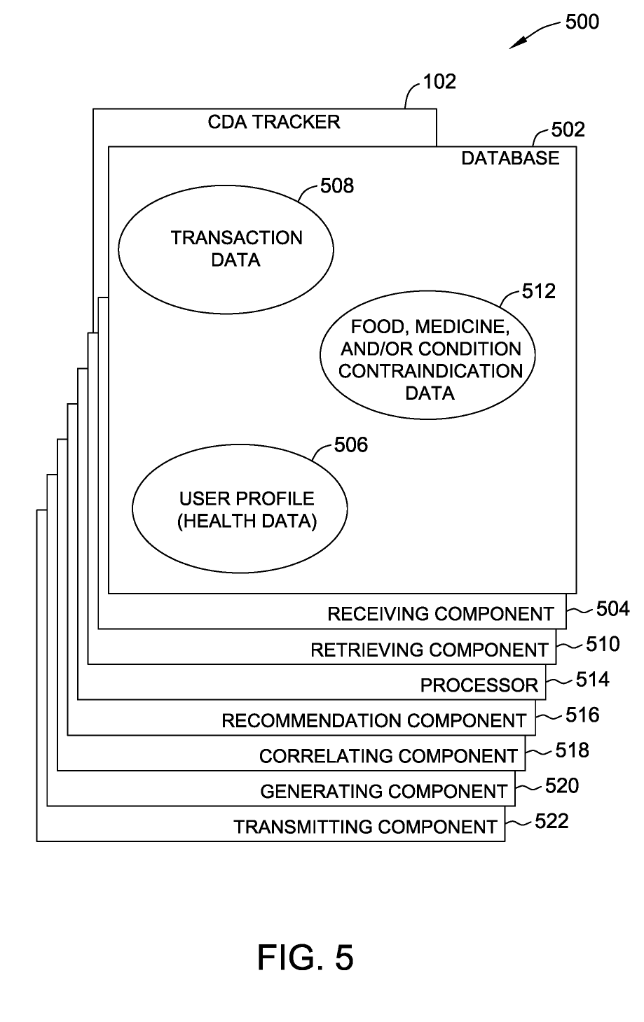

FIG. 5 is a component view 500 of an example CDA tracker 102 (shown in FIG. 1) that may be used in the CDA system 100 (shown in FIG. 1). In the example embodiment, CDA tracker 102 includes a database 502. Database 502 may be coupled with several separate components within CDA tracker 102, which perform specific tasks.

In the example embodiment, CDA tracker 102 includes a receiving component 504. Receiving component 504 may be configured to receive user health data from a user computing device (e.g., user computing device 108, shown in FIG. 1). The user health data may be stored in database 502 in a user profile 506 of the user associated with the user computing device. Receiving component 504 may be further configured to receive transaction data 508 from a payment network, for example, payment network 212 (shown in FIG. 2), and/or merchant computing device 112, wherein transaction data 508 includes, for example, total transaction amounts, time of the transactions, a location of the transactions, and/or line item data (e.g., SKU data). CDA tracker 102 uses the location identifier to locate the user computing device 108, for example, at a merchant location or at home.

CDA tracker 102 further includes a retrieving component 510, which is configured to retrieve available food, medicine, and/or condition contraindication data 512 from, for example, third party open databases 116 (shown in FIG. 1). CDA tracker 102 further includes a processor 514 in communication with a recommendation component 516, a correlating component 518, and a generating component 520. Processor 514 processes the available user profile data 506 (e.g., user health data), transaction data 508, and food, medicine, and/or condition contraindication data 512, to determine one or more personalized indicators indicating at least one contraindication relating to the transaction data 508.

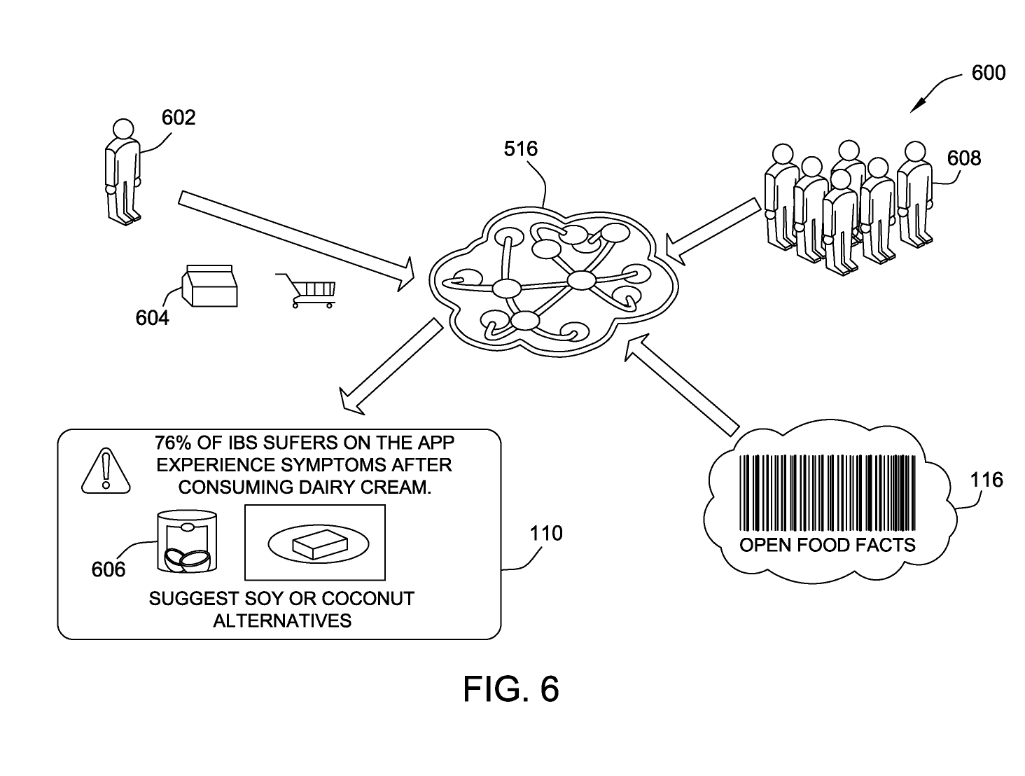

FIG. 6 is a schematic diagram 600 of the process of recommendation component 516. In the example embodiment, after the user opts in to use the CDA tracker service, the CDA tracker provides a seamless user experience with, for example, Masterpass enabled applications such as an e-commerce application. For example, an irritable bowel syndrome (IBS) sufferer 602 is shopping on a Masterpass enabled merchant website or in a merchant store. The IBS sufferer 602 adds dairy cream 604 to her shopping cart (whether virtually or by scanning the item with the user's computing device 108). The recommendation component analyzes the user profile data 506 from other IBS sufferers 608, and the food, medicine, and/or condition contraindication data 512, and determines that dairy cream 604 correlates to symptoms of IBS. Processor 514 (shown in FIG. 5) determines that one or more personalized indicators are needed. Generating component 520 is configured to generate the one or more personalized indicators from the processor 514. A transmitting component 522 of CDA tracker 102 is configured to transmit the personalized indicators to the user computing device 108 for display to the user, for example, within the e-commerce application 110 (shown in FIG. 1). For example, the personalized indicator may indicate to the IBS sufferer that about 76% of other IBS sufferers experienced IBS symptoms after consuming dairy creaming. In addition, the personalized indicator may include a recommendation for a substitute item 606, such as, for example, soy or coconut milk.

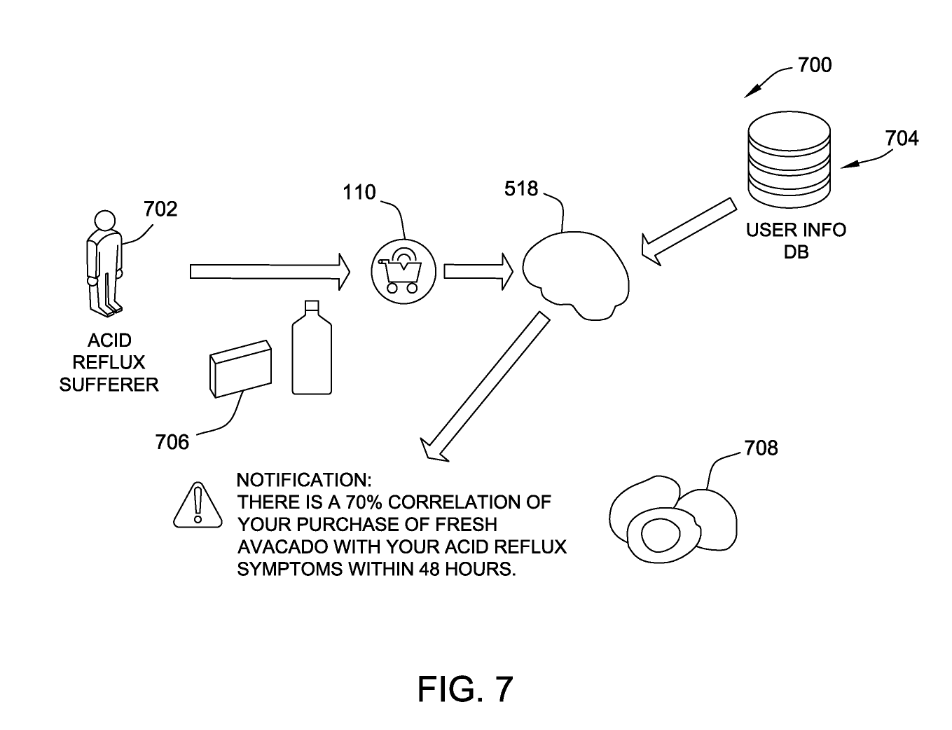

FIG. 7 is a schematic diagram 700 of the process of correlating component 518. In the example embodiment, after an acid reflux sufferer 702 opts in to use the CDA tracker service, processor 514 (shown in FIG. 5) processes the available user profile data 704 (e.g., user health data), transaction data 508 (shown in FIG. 5), and food, medicine, and/or condition contraindication data 512 (shown in FIG. 5). The acid reflux sufferer 702 is shopping on a MASTERPASS enabled merchant website or in a merchant store, and adds avocados 708 to his shopping cart. Correlating component 518 analyzes the available data and correlates acid reflux sufferer's 702 previous purchases of antacids 706 and avocados 708 to determine that the user may suffer from acid reflux. In one embodiment, processor 514 (shown in FIG. 5) determines that one or more personalized indicators may be presented to the user. The personalized indicator may be presented to the acid reflux sufferer 702 when avocados 708 are added to his shopping cart, or as an out-of-band notification. In the example embodiment, generating component 516 (shown in FIG. 5) generates the one or more personalized indicators from the processor 514, and transmitting component 522 transmits the personalized indicator to the user computing device 108 for display to acid reflux sufferer 702, for example, within the e-commerce application 110 (shown in FIG. 1). For example, the personalized indicator may indicate to the acid reflux sufferer 702 that there is a 70% correlation of his purchase of avocados 708 with his acid reflux symptoms with 48 hours of consumption.

In one embodiment, CDA tracker 102 is configured to parse received transaction data not only for the particular products purchased but also for the merchant from whom the products were purchased and/or the particular brand of product purchased. Accordingly, the CDA tracker 102 further determines a usage history associated with the user profile such that the CDA tracker 102 may generate intelligent advice and/or recommendations for the user that accord with their actual item usage preference (e.g., brands, merchants, items). For example, for a user using a virtual shopping cart to generate a shopping list, the CDA tracker 102 may provide a brand corresponding to each product on the list and identify a merchant that the user typically (e.g., most frequently, most recently, on average, etc.) purchases the product from.

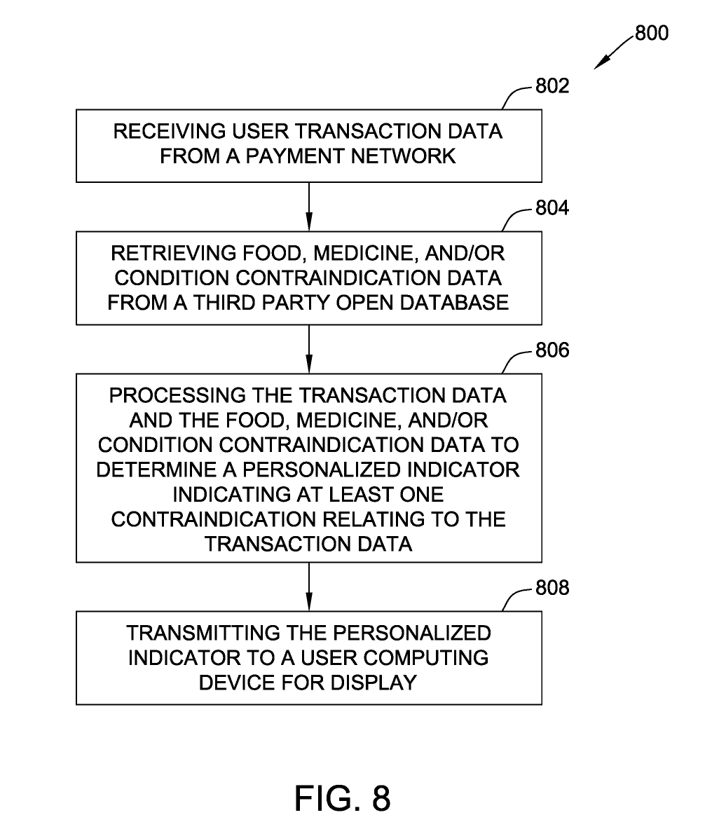

FIG. 8 is a flowchart of a method 800 for tracking food-related transactions of a user and providing advice about food-related and medicinal purchases using the CDA system 100 shown in FIG. 1. Method 800 includes receiving 802 user transaction data (e.g., transaction data 508 (shown in FIG. 5)) from a payment network (e.g., payment network 212, shown in FIG. 1). The transaction data 508 includes, for example, line item data used to identify a user's food-related and medicinal purchases. Method 800 also includes retrieving 804 food, medicine, and/or condition contraindication data (e.g., food, medicine, and/or condition contraindication data 512 (shown in FIG. 5)) from a third party open database, such as third party open database 116 (shown in FIG. 1). The data 512 includes, for example, data related to medical conditions, symptoms, medicines, and foods, and the associated indications and contraindications of such items. Method 800 further includes processing 806 the transaction data 508 and the food, medicine, and/or condition contraindication data 512 to determine a personalized indicator indicating at least one contraindication relating to the transaction data 508. Method 800 also includes transmitting 808 the personalized indicator to a user computing device for display. It is understood that method 800 may include additional, fewer, or different steps, including those described elsewhere herein.

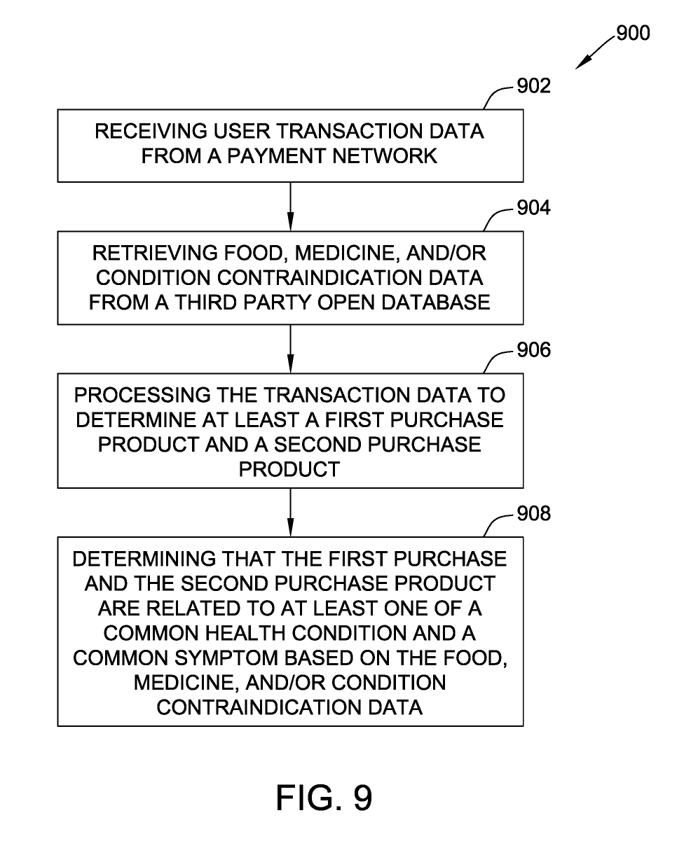

FIG. 9 is a flowchart of a method 900 for tracking food-related transactions of a user and providing advice about food-related and medicinal purchases using the CDA system 100 shown in FIG. 1. Method 900 includes receiving 902 user transaction data (e.g., transaction data 508 (shown in FIG. 5)) from a payment network (e.g., payment network 212, shown in FIG. 1). The transaction data 508 includes, for example, line item data used to identify a user's food-related and medicinal purchases. Method 900 also includes retrieving 904 food, medicine, and/or condition contraindication data (e.g., food, medicine, and/or condition contraindication data 512 (shown in FIG. 5)) from a third party open database, such as third party open database 116 (shown in FIG. 1). The data 512 includes, for example, data related to medical conditions, symptoms, medicines, and foods, and the associated indications and contraindications of such items. Method 900 further includes processing 806 the transaction data 508 to determine at least a first purchased product and a second purchased product. Method 900 also includes determining 908 that the first purchased product and the second purchased product are related to at least one of a common health condition and a common symptom based on the food, medicine, and/or condition contraindication data

As will be appreciated based on the foregoing specification, the above-discussed embodiments of the disclosure may be implemented using computer programming or engineering techniques including computer software, firmware, hardware or any combination or subset thereof. Any such resulting computer program, having computer-readable and/or computer-executable instructions, may be embodied or provided within one or more computer-readable media, thereby making a computer program product, i.e., an article of manufacture, according to the discussed embodiments of the disclosure. These computer programs (also known as programs, software, software applications, or code) include machine instructions for a programmable processor, and can be implemented in a high-level procedural and/or object-oriented programming language, and/or in assembly/machine language. As used herein, the terms “machine-readable medium,” “computer-readable medium,” and “computer-readable media” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium,” “computer-readable medium,” and “computer-readable media,” however, do not include transitory signals (i.e., they are “non-transitory”). The term “machine-readable signal” refers to any signal used to provide machine instructions and/or data to a programmable processor.

In addition, although various elements of the consumption digestion advice tracker are described herein as including general processing and memory devices, it should be understood that the consumption digestion advice tracker is a specialized computer configured to perform the steps described herein for tracking food-related transactions of a user and providing advice about food-related and medicinal purchases.

This written description uses examples, including the best mode, to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A consumption digestion advice (CDA) tracker computing device including a processor coupled in communication with a memory, wherein said processor is programmed to:
receive user transaction data from a payment network for a selected user;
retrieve food, medicine, and/or condition contraindication data from a third party database for the selected user;
process the transaction data and the food, medicine, and/or condition contraindication data to determine a personalized indicator indicating at least one contraindication relating to the transaction data; and
transmit the personalized indicator to a user computing device for display, the user computing device associated with the selected user.

2. The CDA tracker computing device of claim 1, wherein said processor is further programmed to:
receive user health data from the user computing device; and
process the user health data, the transaction data, and the food, medicine, and/or condition contraindication data to determine the personalized indicator.

3. The CDA tracker computing device of claim 2, wherein the user health data includes at least one of a health condition and a symptom of the selected user.

4. The CDA tracker computing device of claim 3, wherein said processor is further programmed to:
analyze the user transaction data to determine at least a first purchased product and a second purchased product; and
determine that the first and second purchased products are related to the at least one of a health condition and a symptom based on the food, medicine, and/or condition contraindication data.

5. The CDA tracker computing device of claim 4, wherein said processor is further programmed to automatically update the user health data.

6. The CDA tracker computing device of claim 5, wherein said processor is further programmed to transmit a health report associated with the selected user to a service provider, wherein the health report includes the updated user health data.

7. The CDA tracker computing device of claim 1, wherein said processor is further programmed to receive line item data corresponding to the user transaction data, the line item data including product-specific-data.

8. The CDA tracker computing device of claim 7, wherein the line item data includes scan data from the user computing device, the scan data including an identifier of a specific product purchased by the selected user.

9. The CDA tracker computing device of claim 7, wherein said processor is further programmed to receive additional health data from the user computing device identifying specific occasions when a medicine successfully treated at least one of a health condition and a symptom.

10. The CDA tracker computing device of claim 1, where said processor is further programmed to transmit the personalized indicator including a recommendation for an alternative product to purchase based on the food, medicine, and/or condition contraindication data.

11. A consumption digestion advice (CDA) tracker computing device including a processor coupled in communication with a memory, wherein said processor is programmed to:
receive user transaction data from a payment network for a selected user;
retrieve food, medicine, and/or condition contraindication data from a third party database for the selected user;
process the transaction data to determine at least a first purchased product and a second purchased product; and
determine that the first and second purchased products are related to the at least one of a health condition and a symptom based on the food, medicine, and/or condition contraindication data.

12. The CDA tracker computing device of claim 11, where said processor is further programmed to:
determine a personalized indicator indicating at least one contraindication relating to the at least a first purchased product and a second purchased product; and
transmit the personalized indicator to a user computing device for display, the user computing device associated with the selected user.

13. The CDA tracker computing device of claim 11, wherein said processor is further programmed to:
receive user health data from the user computing device; and
process the user health data, the transaction data, and the food, medicine, and/or condition contraindication data to determine a personalized indicator indicating at least one contraindication relating to the at least a first purchased product and a second purchased product.

14. The CDA tracker computing device of claim 13, wherein the user health data includes at least one of the health condition and the symptom of the selected user.

15. The CDA tracker computing device of claim 13, wherein said processor is further programmed to automatically update the user health data.

16. The CDA tracker computing device of claim 11, wherein said processor is further programmed to receive line item data corresponding to the user transaction data, the line item data including product-specific-data.

17. A computer-implemented method for tracking food-related transactions of a user and providing advice about food-related and medicinal purchases using a consumption digestion advice (CDA) tracker computing device including a processor coupled in communication with a memory, said method comprising:
receiving user transaction data from a payment network for a selected user;
retrieving food, medicine, and/or condition contraindication data from a third party database for the selected user;
processing the transaction data and the food, medicine, and/or condition contraindication data to determine a personalized indicator indicating at least one contraindication relating to the transaction data; and
transmitting the personalized indicator to a user computing device for display, the user computing device associated with the selected user.

18. The computer-implemented method of claim 17, further comprising:
receiving user health data from the user computing device; and
processing the user health data, the transaction data, and the food, medicine, and/or condition contraindication data to determine the personalized indicator.

19. The computer-implemented method of claim 18, wherein the user health data includes at least one of a health condition and a symptom of the selected user.

20. The computer-implemented method of claim 19, further comprising:
analyzing the user transaction data to determine at least a first purchased product and a second purchased product; and
determining that the first and second purchased products are related to the at least one of a health condition and a symptom based on the food, medicine, and/or condition contraindication data.

21. At least one non-transitory computer-readable storage media having computer-executable instructions embodied thereon, wherein when executed by at least one processor, the computer-executable instructions cause the processor to:
receive user transaction data from a payment network for a selected user;
retrieve food, medicine, and/or condition contraindication data from a third party database for the selected user;
process the transaction data and the food, medicine, and/or condition contraindication data to determine a personalized indicator indicating at least one contraindication relating to the transaction data; and
transmit the personalized indicator to a user computing device for display, the user computing device associated with the selected user.

22. The CDA tracker computing device of claim 21, wherein said processor is further programmed to:
receive user health data from the user computing device; and
process the user health data, the transaction data, and the food, medicine, and/or condition contraindication data to determine the personalized indicator.

23. The CDA tracker computing device of claim 22, wherein the user health data includes at least one of a health condition and a symptom of the selected user.

24. The CDA tracker computing device of claim 23, wherein said processor is further programmed to:
analyze the user transaction data to determine at least a first purchased product and a second purchased product; and
determine that the first and second purchased products are related to the at least one of a health condition and a symptom based on the food, medicine, and/or condition contraindication data.

25. The CDA tracker computing device of claim 24, wherein said processor is further programmed to automatically update the user health data.

26. The CDA tracker computing device of claim 25, wherein said processor is further programmed to transmit a health report associated with the selected user to a service provider, wherein the health report includes the updated user health data.

27. The CDA tracker computing device of claim 21, wherein said processor is further programmed to receive line item data corresponding to the user transaction data, the line item data including product-specific-data.

28. The CDA tracker computing device of claim 21, where said processor is further programmed to transmit the personalized indicator including a recommendation for an alternative product to purchase based on the food, medicine, and/or condition contraindication data.
